(19) United States
(12) Patent Application Publication
(10) Pub. No.: US 2011/0307042 A1
(43) Pub. Date: Dec. 15, 2011

(54) ELECTRODE ARRAYS BASED ON POLYETHERKETONEKETONE

(75) Inventor: Anthony DeCarmine, Lebanon, CT (US)

(73) Assignee: OPM MEDICAL ACQUISITION, LLC, Enfield, CT (US)

(21) Appl. No.: 13/203,654

(22) PCT Filed: Feb. 24, 2010
(86) PCT No.: PCT/US10/25168

§ 371 Date: Aug. 26, 2011

Related U.S. Application Data
(60) Provisional application No. 61/155,699, filed on Feb. 26, 2009.

(51) Int. Cl. A61N 1/05; H05K 13/04

(52) U.S. Cl. 607/116; 29/841

(57) ABSTRACT

Laminated assemblies containing electrode-bearing layers comprised of polyetherketoneketone are useful in the fabrication of implantable medical devices.

### FIELD OF THE INVENTION

The present invention relates to electrodes and more particularly to an electrode array that is relatively thin, conformable and capable of being implanted within the human body. The present invention also provides a method of fabricating such a flexible and implantable electrode array as well as an implantable medical device that includes the inventive electrode array.

### BACKGROUND OF THE INVENTION

In recent years, there has been significant interest in developing electrode arrays for a broad range of applications including, for example, for use in various implantable medical devices. Implantable medical devices are physical articles used in medical treatment that can be introduced into living tissue such as the human body. Examples of implantable medical devices which can contain electrode arrays include, but are not limited to, cochlear implants, visual prostheses, neurostimulators, neuro-prosthetic implants, muscular stimulators, and deep brain stimulators.

A typical electrode array used in an implantable medical device contains multiple electrodes with conducting lines and contacts for interfacing to driving electronics. The conducting lines are used to electrically connect the electrodes of the array to the contacts, which, in turn, are used to interface with the driving electronics of the medical device.

Although a large number of different approaches to the manufacture of such implantable electrode arrays have been described in the literature in recent years, many of these involve multiple, complicated assembly steps. Commercial, cost-effective implementation of these technologies therefore is challenging. Additionally, many different types of implantable devices based on electrode arrays are needed and the conventional assembly methods available currently may not be adaptable to all such desired end-use applications. Consequently, there is still a need for providing additional alternative methods of fabricating flexible, implantable electrode arrays.

A further explanation of the many challenges associated with selecting materials suitable for use in manufacturing implantable medical devices is found in U.S. Pat. No. 6,643,552:
“Microelectronic components, integrated circuits, and implantable electrodes are used extensively in implantable medical devices (IMDs) such as cardiac pacemakers, cochlear prosthesis devices, and neuroprostheses. IMDs can be constructed using a variety of well known methods such as printed circuit boards and hybrid circuits formed on a substrate. Typical hybrid circuits are used can be formed using well known techniques. As the device size and conductor size decrease to below approximately 10 micrometers, the hybrid substrate must be micro-machined using photolithographic techniques to pattern and put down the conductor traces. An IMD needs to be encased with an encapsulant such as silicone that is chemically bonded to the substrate.

Implanting medical devices in a biological environment subjects the IMD to a chemically and electrically harsh environment. For example, the biological environment is highly corrosive to many materials, and the conductors used to connect the device to other electronic circuits or connectors must be able to withstand immersion in an ionic fluid with as much as a 10-volt bias across it. Cardiac pacemakers typically include a hermetically sealed titanium canister containing the power source and associated circuitry and glass sealed electrode feed-throughs to allow the electronic signals generated by the circuitry to interface to the heart muscle. The size of cardiac pacemakers is dominated by the size of the energy source, and typically, the titanium case is a few centimeters in diameter and half a centimeter thick. The leads are typically multi-filament coils of a high nickel content stainless steel alloy and the leads are typically insulated with using silicone. Silicone insulated leads have been very reliable, however, silicone has a tendency to stick to tissue during insertion and to reduce the diameter of the pacemaker leads.

IMDs for neuroprostheses have even more demanding requirements than cardiac pacemakers. Neuroprostheses for rehabilitation of the deaf, blind, spinal cord injured and amputees are being developed that make use of IMDs. In these instances, the IMD requires close proximity to the small and fragile cells of the nervous system. In some cases, the IMDs will be attached or embedded directly in the neural tissue. The neural tissue is a very dynamic environment, for example peripheral nerves stretch and relax with the motion of a limb, the spinal cord moves within the spinal canal, the brain moves relative to the skull any motion of the head and also with each heartbeat, and movement of the eyes creates substantial acceleration forces on the retina.

Because of the nature of the biological environment, the fragile nature of the neural tissue, the high packing density of the neural tissue, the effects of dissimilar acceleration, and the proliferation of connective tissue that can encase an IMD, IMDs used as neuroprostheses must be biocompatible, bioresistant, be of small size, be density matched to the surrounding neural tissue and be minimally tethered to the surrounding tissue.

Biocompatibility is essential in an IMD to minimize the formation of connective tissue between the nearby neurons and the IMD over the course of long term or chronic implantation. Bioresistance, or chemical inertness with respect to the biological environment is essential to prevent corrosion from damaging the IMD. An IMD needs a small size to minimize damage to the target neural structures during implantation.

To avoid differential acceleration between the IMD and the surrounding tissue, matching the density of the two is important to avoid damage to the surrounding tissue. Minimal tethering between an electrode and an electronic device will reduce the transmission of forces transmitted along the wiring between the electronic device and the implanted electrode, particularly after being encased in connective tissue as part of the normal healing process.

Silicon has been the material of choice for neuroprosthetic IMDs because of its mechanical and chemical properties. For example, silicon can be micro-machined to extraordinarily small dimensions, is very strong, relatively corrosion resistant, can have embedded integrated circuits for signal processing or controlling functions, and because it forms an inert self limiting oxide that is biocompatible. Silicon may be micro-machined to produce a variety of novel structures. Silicones are an important class of materials that can both insulate silicon substrates as well as protect silicon substrates from corrosive environments. However, although silicone has been shown useful as an encapsulant, silicone has not been useful as a micro-machined substrate because it is not dimensionally stable and thus cannot support fine metal patterns or be photolithographically processed. Of the many candidate materials that have been used in the prior art, only polyimide was a possible polymer that could be used for flexible implantable microelectrode array substrates. Polyimide has been used extensively by researchers for producing microelectrode arrays for cochlear electrode arrays, retinal prostheses, peripheral nerve electrodes, and central nerve electrodes. While polymer based flexible electrodes have been previously developed using polyimide, polyimide is not a very long-term water resistant material. Furthermore, polyimide is used as a sensor for humidity because of its hygroscopic quality. Although polyimide structures may be able to withstand up to several years of static immersion in saline, the failure modes of polymide structures are usually linked to mechanical weakening of the material due to hydrolytic attack.

Micro-machined silicon substrates as fabricated are not bioresistant and can have multiple failure modes when an integrated circuit or microelectronic hybrid circuit are formed thereon. The wires used to attach to the circuit elements must be able to withstand immersion in ionic fluids. Exposed areas where the wires are attached to connectors or devices are coated with encapsulant material that is applied after wire bonding as been accomplished. If micro-ribbon technology is used, it is necessary to create a void free seal in the area under the micro-ribbon attached to the device. Circuits on the chip must be protected from water and ionic contamination and the chip substrate and encapsulants must be bioresistant and biocompatible.”

Thus, it is clear there is still a need to develop electrode arrays to be incorporated into implantable medical devices that utilize biocompatible and biostable substrates and/or encapsulants.

### SUMMARY OF THE INVENTION

A laminated assembly, which preferably is adapted to be implanted in a subject, is provided by the present invention, wherein the laminated assembly comprises an electrode-bearing layer comprised of polyetherketoneketone.

The present invention provides an alternative approach for fabricating an electrode array that has a laminate-type structure and utilizes a layer comprised of polyetherketoneketone as an electrode-bearing surface. The laminate may comprise one or more additional layers also based on polyetherketoneketone.

The electrode-bearing layer comprised of polyetherketoneketone may include a plurality of patterned conductive features comprising conductive contacts, conductive lines (traces) and conductive electrodes to provide an electrode array on a surface of such layer, which in one embodiment is a thin film. The conductive features may be comprised of metal, conductive polymer, conductive carbon or any other conductive material. In accordance with the present invention and within the array, a single conductive electrode may be contacted to a single conductive contact by a single conductive line. The conductive contact may be a contact pad or other such feature which permits the array to be interfaced with still further components, such as a multiplexer, signal processing component, chemical sensor, photoelectric sensor, signal transmitter, signal receiver, or the driving electronics. The conductive contact may be incorporated into a connection element (connector), to facilitate connection to other components such as a testing or measurement device or stimulation source. In some embodiments of the present invention, it is possible that there could be more than one electrode associated with a single contact. The polyetherketoneketone layer thus functions as a support for the electrode array.

The use of polyetherketoneketone is advantageous in that excellent bonding can be directly achieved between the surface of such layer and the various components comprising the electrode array (especially where such components are metallic), without the need to use separate adhesive materials or to specially treat or condition the polymer surface or the conductive features. Polyetherketoneketone films and sheets are particularly heat resistant and dimensionally stable, exhibiting little tendency to curl when heated, for example, which renders such materials well suited for fabrication processes involving exposure to elevated temperatures. Assembly and delamination problems are thus reduced, as compared to other materials which might be used to support the electrode array. Additionally, polyetherketoneketone is biocompatible and biostable and exhibits particularly good mechanical properties, making it possible to fabricate films that are thin but nonetheless flexible and strong. Polyetherketoneketone has the following further advantages which make it well suited for use in the fabrication of implantable electrode array assemblies in accordance with the present invention: low dielectric constant, exceptional resistance to solvents and water, low moisture uptake, and good wear and radiation resistance. Additionally, although thin films of polyetherketone are capable of being deflected or conformed without breaking or cracking, they are sufficiently stiff to allow an assembled electrode array fabricated from such films to be easily inserted into the desired position within the body (e.g., inserted through a small incision in a subdural application) without folding or doubling.

A second polymeric layer, that is also implantable and biocompatible and that in one embodiment of the invention is also comprised of polyetherketoneketone, can be formed on the electrode-bearing layer (by lamination of a film, for example) such that certain of the patterned conductive features are encapsulated (i.e., surrounded or encased) within the polymeric layers. The second polymeric layer may be pre-patterned prior to forming on the electrode-bearing layer or the second polymeric layer may be patterned after application to the electrode-bearing layer. The patterns formed into the second polymeric layer are typically vias (i.e., openings) that extend down to certain of the first patterned conductive features exposing, for example, the conductive contacts and/or conductive electrodes. The patterns also define the shape of the electrode array. The vias can be filled with a conductive material and contacts can be made between such conductive material and other elements or components of an implantable medical device.

In addition, the present invention can also provide an electrode array that is useful in implantable medical devices. The inventive electrode array includes at least first and second implantable and biocompatible polymeric layers, with at least one of such layers being comprised of polyetherketoneketone, in which a plurality of patterned conductive features including, for example, conductive (e.g., metallic) contacts, conductive lines and/or conductive electrodes and possibly one or more other components such as connectors, amplifiers, photosensors, chemical sensors, light emitting diodes, multiplexers (including de-multiplexers), signal conditioners or other types of signal processing devices is sandwiched therebetween.

In addition to the array, the present invention also provides an implantable medical device which comprises at least the electrode array of the present invention.

### BRIEF DESCRIPTION OF THE DRAWINGS

FIG. 1 (not to scale) is a perspective view of an illustrative electrode-bearing layer in accordance with the invention.

FIG. 2 (not to scale) is a perspective view of an illustrative laminated assembly containing an electrode-bearing layer in accordance with the invention.

### DETAILED DESCRIPTION OF THE INVENTION

The polyetherketoneketones suitable for use in the present invention comprise (and preferably consist essentially of) repeating units represented by the following formulas I and II:

—A—C(═O)—B—C(═O)— I

—A—C(═O)—D—C(═O)— II

where A is a p,p′—Ph—O—Ph— group, Ph is a phenylene radical, B is p-phenylene, and D is m-phenylene. Although the Formula I: Formula II (T:I) isomer ratio in the polyetherketoneketone can range from 100:0 to 0:100, in one embodiment a polyetherketoneketone having a T:I isomer ratio of from about 50:50 to about 90:10 is employed. In one embodiment, the polyetherketoneketone is semicrystalline. In another embodiment, the polyetherketoneketone is amorphous. Amorphous polyetherketoneketone can be easily extruded into a film, self-adheres, and is also capable of being readily vacuum-formed.

Polyetherketoneketones are well-known in the art and can be prepared using any suitable polymerization technique, including the methods described in the following patents, each of which is incorporated herein by reference in its entirety for all purposes: U.S. Pat. Nos. 3,065,205; 3,441,538; 3,442,857; 3,516,966; 4,704,448; 4,816,556; and 6,177,518. Mixtures of polyetherketoneketones may be employed.

Suitable polyetherketoneketones are available from commercial sources, such as, for example, the polyetherketoneketones sold under the brand name OXPEKK by Oxford Performance Materials, Enfield, Connecticut, including OXPEKK-C, OXPEKK-SP, OXPEKK-MG and OXPEKK-IG polyetherketoneketone. Implantable and/or medical grades of polyetherketoneketone are especially useful in the present invention.

The polyetherketoneketones suitable for use in the present invention are thermoplastic materials which can be formed into films, sheets and other desired shapes by standard processing methods such as melt extrusion. Conventional single screw or twin screw extruders, sheeting dies, and take-up devices designed for extrusion of thermoplastic resins into sheets and films are satisfactory. Quenching and/or drawing and/or orientation of the film following extrusion may be practiced, if so desired. For example, the polyetherketoneketone polymer in the form of powder, flakes, or pellets or the like is fed to a conventional plastics extruder, either single or twin screw, wherein the polymer is thoroughly melted and conveyed to a film extrusion die wherefrom it can be extruded onto a quench drum and thence conveyed by a series of guides to a wind-up. The extrusion temperature will depend upon the melt temperature of the polyetherketoneketone, which is influenced by the T/I ratio as well as on the molecular weight or melt viscosity of the polymer. In general, extrusion temperatures from about 10 degrees C. to about 50 degrees C. above the melting point of the polyetherketoneketone will be sufficient.

Methods of forming polyetherketoneketones into films, sheets and the like are well-known in the art and are described in, for example, U.S. Pat. No. 3,637,592; U.S. Pat. No. 4,996,287; GB 1,340,710; and U.S. Pat. No. 5,049,340, each of which is incorporated herein by reference in its entirety.

For purposes of the present invention, it will generally be desirable to form the polyetherketoneketone into films that are relatively uniform in thickness and from about 0.001 to about 0.005 inches (about 25 to about 125 microns) thick, although other types of polyetherketoneketone substrates could also be used. The width, length and overall shape of the polyetherketone film may be varied as desired to suit particular end-use applications and desired electrode array configurations and designs. The film may be cut or shaped from a continuous roll of material using any of the conventional methods known in the art, such as slitting, die cutting and the like. Such cutting and shaping methods can also be employed after placing or creating the desired patterned conductive features on the surface of the polyetherketoneketone film and laminating such conductive feature-bearing film to another material, such as a second polyetherketoneketone film.

The polyetherketoneketone may, if so desired, be compounded or formulated with any of the customary types of additives known or used in the thermoplastic art, including, for example, fillers, pigments, stabilizers, processing aids, and the like.

The present invention, which provides a method of fabricating flexible and implantable electrode arrays as well as the electrode arrays themselves, will now be described in greater detail by referring to the following discussion and drawings that accompany the present application. The drawings which are included with the present application are provided for illustrative purposes and, as such, they are not drawn to scale.

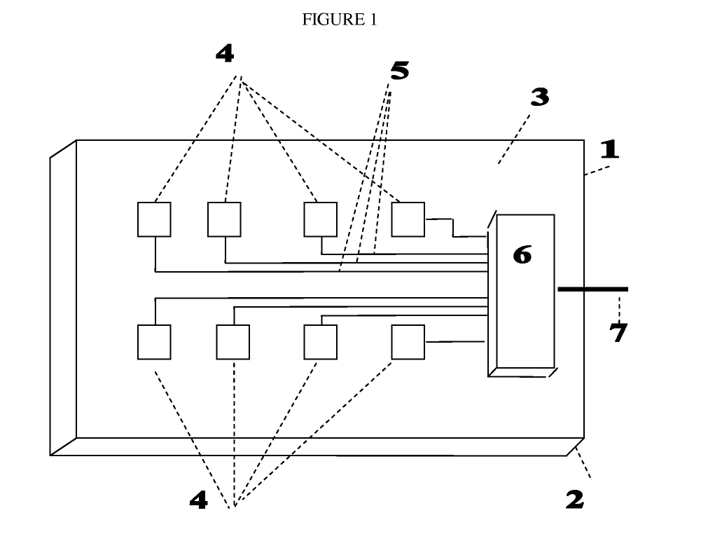

FIG. 1 shows an electrode-bearing layer 1 comprised of a polyetherketoneketone film 2 and, on a surface 3 thereof, a plurality of conductive features including metallic electrodes 4 and metallic lines 5. The term “conductive features” as used in this application includes conductive electrodes, conductive lines, and/or conductive contacts, although other types of conductive features may also optionally be present. In accordance with the present invention, the conductive lines provide electrical contact between the electrodes and other components of the assembly, such as conductive contacts (which can be in the form of metallic pads, for example). It should be noted that more than one electrode can be associated with a single contact. Conductive lines 5 lead to multiplexer 6. Multiplexer 6 (which can be electrically connected to multiple metallic contacts) may be configured so as to enable the individual conductive lines 5 and conductive electrodes 4 to be electrically connected with one or more components located outside the laminated assembly of the present invention, such as the electronics which drive the laminated assembly. The use of a multiplexer enables the number of lines or wires leading from the laminated assembly to be reduced or minimized. In FIG. 1, for example, a single paired lead 7 (which may be a metallic wire, for example) electrically connects multiplexer 6 to another component (not shown), which together with laminated assembly 1 may be part of a fully implantable medical device placed entirely within a human body or which may be external to the human body (with the laminated assembly 1 being located within a human body).

The electrodes may be of any desired shape, size or configuration, depending upon the intended end-use for the laminated assembly and implantable medical device containing such laminated assembly. For example, the electrodes may be microelectrodes that are quite small in size, making it possible to provide a compact electrode array with a high electrode density. The electrode-bearing layer preferably provides an array of electrodes, which can range in number from two to dozens, hundreds or even thousands of electrodes. The electrodes can be designed and operated such that they are either sensor electrodes (i.e., capable of sensing, detecting or measuring signals) or effector electrodes (i.e., capable of delivering signals or electrical stimulation). The electrode array can be linear, a rectangular grid, or any other suitable arrangement.

The laminated assembly of the present invention may comprise an amplifier, signal conditioner, or other signal processing component (e.g., filtering circuitry, noise reduction circuitry), with such signal processing component being advantageously encapsulated between the layers of the assembly. The positioning of such signal processing component within the laminated assembly is especially desirable where the signals being processed are relatively small or weak. Amplification or signal conditioning at or near the point of detection is useful in such situations, since the noise pickup from even a relatively short lead can make it difficult to detect the weak original signal from a location remote from the detection point.

As an example, a brain-computer interface may be provided which comprises an implantable neurochip adapted to communicate with the implantable electrode array and to filter and/or amplify neural signals. After filtering and amplification, the neurochip can multiplex the neural signals and transmit the signals to a data acquisition unit. The mechanism of the transmission can be via a cable or by telemetry or other wireless manner. Preferably, a neurochip is adapted to transmit the signals via telemetry because when this manner of transmission is employed, the skull and scalp of the patient can be fully closed, permitting an implanted neurochip and electrodes to operate without the need for the patient to be physically associated with the data acquisition unit. This approach also minimizes the chances of infection and other undesired conditions.

Electrode-bearing layer 1 may be fabricated using a variety of possible different techniques.

In one embodiment, a hot stamping or hot embossing method is used to directly form the desired patterned conductive features on the surface of a polyetherketoneketone film. Such methods are well known in the printed circuit board art and are described, for example, in U.S. Pat. No. 4,495,232 and U.S. Pat. Pub. No. 2002-0018880, both of which are incorporated herein by reference in their entirety for all purposes. These methods typically use what is referred to as a “stamping foil”, which in addition to a metallic layer (which is of the thickness desired for the conductive feature, e.g., about 5 to about 100 microns) that is to be transferred to a substrate surface to form the desired patterned conductive feature(s) often also includes a carrier (which is used to support the relatively thin metallic layer and facilitate its handling) as well as a separating or release layer between the metallic layer and the carrier to facilitate release of the metallic layer from the carrier upon activation. Although it is common for such stamping foils to also include a bonding or adhesive layer to bond the metallic layer to the substrate surface when applied, an advantage of the present invention is that polyetherketoneketone inherently exhibits good adhesion to metal surfaces, thus making the use of a separate adhesive unnecessary. The stamping foil is positioned against the substrate surface and then activated in a selective pattern by compressing with a stamping die or stereotype, whereby the metallic layer becomes bonded to the substrate surface and separated from the carrier in the preselected activated areas. Heat preferably is also applied (through the use of a heated stamping die, for example) so as to further enhance the bonding achieved. The non-activated portions of the metallic layer remain on the carrier, which is then separated from the substrate surface. Preferably, the metallic layer has relatively low shear strength, to permit sharp, precise separation of the activated areas from the unactivated areas of the stamping foil.

In another embodiment, the plurality of conductive features can be formed by providing a laminate comprised of a metallic foil or sheet and a polyetherketoneketone film and patterning the foil or sheet by laser cutting or wet chemical etching or other suitable technique.

The term “metallic” is used in the present invention to denote a material that includes at least one metal or metal alloy that is electrically conductive. Illustrative examples of metallic materials that can be used in forming the plurality of conductive features include, but are not limited to, gold (Au), silver (Ag), platinum (Pt), copper (Cu), aluminum (Al), titanium (Ti), nickel (Ni), chromium (Cr), tungsten (W), tantalum (Ta), rhodium (Rh), alloys of such metals as well as metals that have been modified with non-metallic elements or substances. In one embodiment of the present invention, Pt or a platinum-containing material (such as Pt/10% Ir) is used as the metallic material of the least one conductive feature. It is noted that in the present invention a single metallic foil or sheet can be used to provide the plurality of conductive features which is an advantage over some of the prior art in which multiple foils or sheets are used in creating such features.

The thickness of the metallic foil or sheet may vary and can be determined by the skilled artisan. Typically, the metallic foil or sheet used will have a thickness from about 5 to about 500 microns, with a thickness from about 10 to about 75 microns being more typical.

The metallic foil or sheet may be united with the polyetherketoneketone film to form a unitary, laminar structure, preferably without using any adhesive agent between the layers. This may be accomplished, for example, by melt-extruding the polyetherketoneketone as a film directly onto the metallic foil or sheet. Following extrusion of the polymer onto the metallic foil or sheet and before complete cooling of the extruded polymer, the laminate can be advanced through one or more nip rollers under pressure to bring the layers into firmer contact to enhance the bonding between the layers.

Alternatively, the aforedescribed laminate may be manufactured by pressure laminating a preformed film of polyetherketoneketone to the metal foil or sheet. Such an operation may be advantageously carried out at a temperature effective to soften the polyetherketoneketone film, with pressure being applied to ensure good adherence of the polymer to the metal. Hot embossing methods known in the art may be adapted for this purpose. Heated pressure rollers may be employed, for example. The lamination can be performed either with individual sheets of the two materials or continuously with rolls of the two materials.

If so desired, the surface of the polyetherketoneketone film to be joined to the metallic foil or sheet is treated prior to bonding to activate the polymeric surface. For example, the polyetherketoneketone surface may be subjected to oxygen plasma treatment, flame treatment, corona treatment or the like. However, one advantage of the present invention is that such pretreatment steps generally are not needed, as the polyetherketoneketone inherently exhibits very good adhesion to metallic surfaces even in the absence of any modification.

The surface of the metallic foil or sheet to be joined to the polyetherketoneketone should be free of dust, oil and other contaminants. In one embodiment of the invention, the metallic surface is modified or pretreated so as to further enhance the degree of bonding achieved between the metallic foil or sheet and the polyetherketoneketone film. Suitable metal surface treatments include, for example, oxidation, conversion coating and the like.

After joining the metallic foil or sheet to the surface of the polyetherketoneketone film, the plurality of conductive features can formed by laser machining, for example. Laser machining is generally a technique that is used in fabricating medical stents and is thus well known in medical device fabrication. Typically, laser machining is performed utilizing a laser system that is scanned over a substrate, ablating material where the laser energy contacts the substrate.

In another embodiment of the present invention, the plurality of conductive features is formed utilizing photolithography and etching. The term “photolithography” is used in this application to denote a patterning technique in which a photoresist (either positive-tone or negative-tone) is applied to the upper exposed surface of a metallic foil needing patterning. The photoresist can be applied by utilizing any deposition technique, with spin-on coating, dip-coating, and spray coating being highly preferred. Following the application of the photoresist, the photoresist is exposed to a pattern of radiation. In the present invention, the pattern of radiation allows for the formation of the plurality of conductive features. After radiation exposure, the exposed resist is developed utilizing a conventional resist developer. The lithographic step thus forms a patterned photoresist having the pattern of the plurality of conductive features located therein. This pattern is then transferred to the metallic foil utilizing an etching process. The etching process may include a dry etching technique such as, for example, reactive-ion etching (RIE), ion beam etching, plasma etching or laser ablation. Alternatively, the etching can be achieved utilizing a chemical wet etching process in which a chemical etchant that selectively removes the exposed portions of the metallic foil is used. After pattern transfer via etching, the patterned photoresist is removed utilizing a conventional stripping process well known to those skilled in the art.

By way of further explanation, conductive features may be formed from the metallic foil which has been laminated to the polyetherketoneketone by covering the areas of the foil or sheet in the laminate where the conductive features are desired with a resist which is waterproof, leakproof, and not attacked by the etchant to be used. Suitable resists, for example, include waxes, resins, inks and the like. The areas of bare metal are then removed with an etchant which attacks the metal. Typically, the etchant (which often is in the form of an aqueous solution) dissolves the metal or chemically reacts with the metal and converts it into a soluble species. After the desired degree of etching is realized, the laminate is washed free of etchant and the resist removed by any appropriate method (for example, waxes, resins and inks can be removed by washing with a solvent suitable for the specific resist material used).

The conductive features, in particular the conductive lines (which are generally relatively thin and narrow), may also be formed by other techniques, such as for example the use of electrically conductive inks, pastes or the like which can be applied in the desired locations on the polyetherketoneketone film surface by spraying, painting, jet printing, transfer printing and the like. A mask can be used to control the placement of the conductive features.

For example, a metal- or metal precursor-containing ink such as an ink containing silver or gold particles (e.g., nanoparticles) or silver nitrate may be applied to the polyetherketoneketone film surface in an imagewise fashion using ink jet printing and screen printing methods known in the art. Other printing technologies such as gravure, offset, micro-contact, pad or flexographic printing as well as nano-contact lithography could also be utilized. Once the ink is applied in the desired locations and pattern, further treatment of the printed film may be carried out to form the desired conductive features (for example, the metal precursor in the ink may be converted to metal or any solvent or volatile carrier present in the ink may be removed by heating or other drying procedure). One suitable printing method is described by Professor Jennifer Lewis of the University of Illinois and her colleagues in an article entitled “Omnidirectional Printing of Flexible, Stretchable, and Spanning Silver Microelectrodes” in the Feb. 12, 2009, edition of Science. A highly concentrated silver nanoparticle ink is extruded through a tapered cylindrical nozzle attached to a three-axis micropositioning stage, which is controlled by computer-aided design software. When printed, the silver nanoparticles are not yet bonded together. The bonding process occurs when the printed structure is heated to 150 degrees C. or higher. During thermal annealing, the nanoparticles fuse into an interconnected structure. Polyetherketoneketone is particularly well suited for use in this type of printing process, since films and sheets comprised of polyetherketoneketone exhibit exceptional dimensional stability and thus little tendency to curl or distort when heated.

The conductive features may alternatively be comprised of a conductive material other than metal, such as, for example, a conductive polymer or carbon particles (e.g., carbon nanotubes, graphite). Conductive polymers are well known in art and include polyacetylenes, polypyrroles, polythiophenes, polyanilines, polyfluorenes, poly(3-alkylthiophene)s, polytetrathiafulvalenes, polynaphthalenes, poly(p-phenylene sulfide)s, and poly(para-phenylene vinylene)s. Typically, the polymer will be doped or otherwise modified to impart or enhance its conductive properties, although inherently conductive polymers can also be used.

In another approach, one or more of the conductive features may be first formed on a carrier substrate and then transferred to the polyetherketoneketone film.

The carrier substrate may be in the form of a flat sheet or wafer comprised of Si, glass, plastic, ceramic or the like. In one embodiment of the present invention, a non-stick layer can be applied to the carrier substrate prior to forming the conductive features thereon so as to facilitate the desired later transfer of the conductive features to the surface of the polyetherketoneketone film. The conductive features may be formed on the carrier substrate using any suitable method, including the methods described previously in connection with forming the conductive features directly on the polyetherketoneketone film. Once the conductive features have been created, the surface of the carrier substrate bearing such features is brought into intimate contact with the surface of the polyetherketoneketone film under conditions effective to cause transfer of the conductive features from the carrier substrate to the polyetherketoneketone film (pressure and/or heat may be applied, for example). The carrier substrate is thereafter separated to provide the electrode-bearing layer.

Alternatively, the conductive features may be formed on the surface of the polyetherketoneketone film or the carrier substrate by placing a thin coating of metal on such surface by vacuum deposition, physical vapor deposition (PVD), chemical vapor deposition (CVD) or sputtering through a photomask, with subsequent electroplating optionally being used to increase the thickness of the metal coating. The conductive features can then be created from the metal coating by photolithographic etching means. It is also possible to avoid having to use etching techniques by using methods capable of applying the metal only in the desired pattern and location directly to the film or substrate surface.

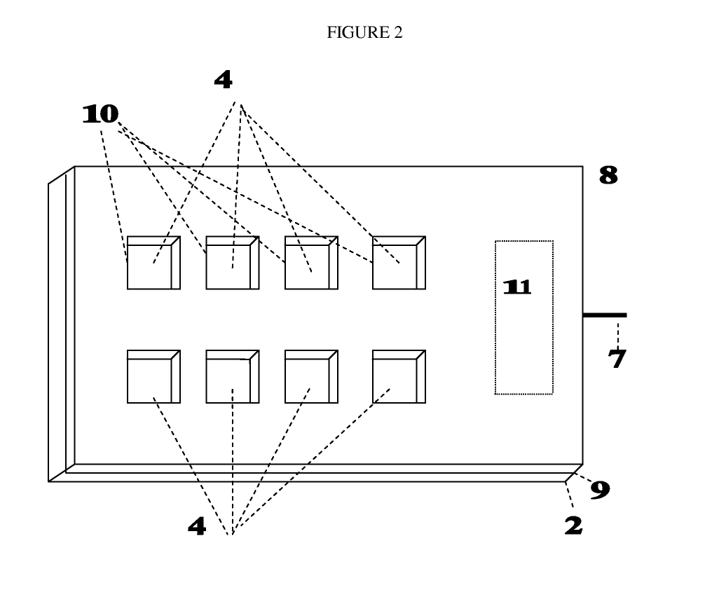

FIG. 2 shows a laminated assembly 8 after forming a top layer 9, which is preferably comprised of an implantable and biocompatible polymer, on the electrode-bearing layer such that the plurality of conductive features as well as the multiplex are surrounded, i.e., encased, within the two layers. Such encapsulation helps to ensure the electrical functionality of the laminated assembly (implanted electrodes and the conductive lines connected to them must be very effectively insulated, because of the very small voltages and currents being utilized) and protects the laminated assembly against the harsh environments that can be encountered within the body (e.g., saline fluids, blood, acidic fluids).

The top layer 9 is most preferably comprised of polyetherketoneketone so as to provide good compatibility and adhesion between the layers of the assembly 8, but could alternatively be comprised of a different polymeric material. As shown, top layer 9 includes a plurality of vias 10 which extend down through top layer 9 and provide contact openings where a conductive material can be formed. The vias 10 expose some of the underlying conductive features, e.g., the conductive electrodes 4 or contacts but leave covered other conductive features such as conductive lines 5. Thus, the vias 10 are present in preselected locations within the laminated assembly 8. Multiplexer 6 is encased within region 11 of top layer 9. As the multiplexer or other features or components present between the electrode-bearing layer (such as a signal conditioner or amplifier) and the top layer may be thicker than certain other features (such as the conductive electrodes and conductive lines), it may be advantageous to shape the top layer prior to laminating it to the electrode-bearing layer so as to provide preformed depressions or spaces in selected areas of the top sheet to accommodate such thicker components. This can be done by thermoforming or vacuum forming of the top layer.

The laminate assembly shown in FIG. 2 can be formed by applying top layer 9 to the structure shown, for example, in FIG. 1. Top layer 9 can be deposited utilizing one of the above mentioned lamination processes that was used in forming the composite of the polyetherketoneketone film and the metal foil or sheet. For example, top layer 9 may be preformed as a film and then laminated to the electrode-bearing layer 1 (using heat and/or pressure, e.g., in a hot embossing or stamping process) or extruded directly onto the electrode-bearing layer 1. In one desirable embodiment, top layer 9 and polyetherketoneketone film 2 are thermally fused to each other. For example, top layer 9 and polyetherketoneketone film 2 may be conglomerated into a single continuous film, having no boundaries between layers (except, of course, in those areas where conductive features or other components of the laminated assembly are sandwiched between top layer 9 and polyetherketoneketone film 2), by heat treatment. Preferably, no adhesive is used to bond top layer 9 and polyetherketoneketone film 2. One advantage of the present invention is that it is possible to fabricate implantable electrode arrays that contain no adhesive, thereby reducing or eliminating any adhesive failure issues or biocompatibility or biostability issues that might otherwise be created by the presence of adhesives. The vias 10 can be formed into top layer 9 by photolithography and etching or by laser machining, before or after laminating top layer 9 and electrode-bearing layer 1.

For example, the structure shown in FIG. 2 can be formed by providing a pre-patterned top layer 9 that contains said vias on a carrier substrate. This pre-patterned structure is formed by first applying top layer 9 to a carrier substrate and subjecting top layer 9 to photolithography and etching or laser machining. This structure is then adhered to the structure shown in FIG. 1 utilizing the bonding conditions mentioned above. Die cutting may also be used to create a pre-patterned top layer 9, where openings in the top layer 9 are formed by dies.

A conductive material (comprised, for example, of metal or a conductive polymer) can be filled into the vias 10 utilizing a conventional deposition process. Following deposition, any conductive material outside the vias can be removed utilizing a conventional planarization process. The filled vias allow for the laminated assembly 8 shown in FIG. 2 to be interfaced with other components of an implantable medical device including, for example, an energy source and/or a sensor or allow the electrodes to come into more direct electrical contact with a surface with which the electrode array is brought into contact (e.g., the surface of an organ). The sidewalls of the vias provide openings to make contact to the electrodes.

The laminated assembly may be further comprised of additional layers, if so desired. In one embodiment, the additional layer is comprised of polyetherketoneketone (e.g., a polyetherketoneketone film), although other materials (especially biocompatible and biostable materials) could also be used. For example, an additional polyetherketoneketone film may be adhered to the bottom surface of polyetherketoneketone film 2 in FIG. 2. Additional conductive features or other electronic or electrical components may be sandwiched between these film layers. One or more vias containing conductive material may extend down through polyetherketoneketone film 2 and electrically connect with such additional conductive features or other electronic or electrical components with conductive features or other components on the top surface of polyetherketoneketone film 2.

As stated above, the inventive laminated assembly is suitable for use as a component in an implantable medical device. Such implantable medical devices include, for example, cochlear implants, visual prostheses, neurostimulators, neuro-prosthetic implants, muscular stimulators, pulse generators, brain-computer interfaces and deep brain stimulators. Implantable medical devices in accordance with the invention are useful as neural protheses to restore body function after paraplegia by means of functional electrical stimulation (FES). The electrode arrays provided by the present invention are particularly useful where surface contact with an organ or other living tissue is needed so as to provide measurement of activity within that organ or tissue (where the electrodes act as sensors) and/or stimulation of activity within that organ or tissue (where the electrodes act as effectors). The electrode array may take any suitable form or shape as may be needed for a particular desired end-use application. For example, the laminated assembly may be relatively long but narrow in width, enabling it to be inserted into a confined space within the body (within the spine space, for instance) without the need to create a large opening in the body in order to place the electrode array where desired to provide telemetry or to bypass injury and/or restore function.

The implantable medical device may be fully implantable, i.e., all components needed to operate the device are contained within the body. In this embodiment, the medical device may be configured and designed so as to be capable of transmitting and/or receiving signals remotely (without the use of wires or leads). Alternatively, the electrode array may be implanted, with the electronics needed to operate the array (such as an electronic stimulator device) being maintained external to the body and physically connected to the array by wires, electrical leads or the like. Although the laminated assembly of the present invention is specifically suitable for use in an implantable medical device, it can also find uses in electronic devices other than implantable medical devices. Other applications for the inventive laminated assembly include, but are not limited to: electrodes and electrical interconnects for medical devices that are not implanted, consumer electronics subjected to water immersion or splashing, sensor arrays (for chemical processes and harsh, high temperature and/or corrose environments, for example) and underwater sensing systems.

The lamination can be manufactured, using the aforementioned methods, to include aligned sets of vias (holes) that cause a planned hole through the item, which can be used for fixation to living tissue by suture, screw or other method. Such fixation may be applied to another synthetic device also implanted in the vicinity for related or unrelated therapeutic purposes by the aforementioned methods or by the use of a feature incorporated into the neighboring device. A noteworthy feature of the invention is that it is possible, if the specific design of an implementation allows, for the surgeon to make additional fixation holes during the procedure with instruments readily available.

Further, said fixations vias need not penetrate through the device—an array of small holes, penetrating only the outermost layer(s), could be provided to create a surface providing added mechanical interlock for direct friction or as an aid to adhesives sometimes used in such procedures.

The present invention provides several advantages over prior art electrode arrays. First, the invention makes possible the fabrication of electrode arrays with relatively robust conductive features that are flexible and rugged. Moreover, the metallic contacts, lines, electrodes, and other conductive features can be made using a single continuous metallic sheet or foil, thereby simplifying the overall assembly process and eliminating potential problems associated with depositing or forming multiple conductive metal films or layers. The fabrication process provided by the present invention is simple and low cost, and facilitates the manufacture of electrode arrays containing hundreds or even thousands of electrodes. The invention takes advantage of well-characterized manufacturing techniques (such as, for example, film/foil lamination, laser machining, photolithography, hot embossing, transfer printing, ink jet printing, screen printing) and lends itself well to mass production.

What is claimed:

1. A laminated assembly comprising an electrode-bearing layer comprised of polyetherketoneketone.

2. The laminated assembly of claim 1, wherein said electrode-bearing layer is a polyetherketoneketone film having one or more metallic electrodes adhered thereto.

3. The laminated assembly of claim 2, wherein said polyetherketoneketone film has a thickness of from 0.001 to 0.005 inches.

4. The laminated assembly of claim 1, wherein said electrode-bearing layer is a polyetherketoneketone film which supports a plurality of conductive features, said conductive features being comprised of one or more conductive materials selected from the group consisting of metals, conductive polymer, and conductive carbon.

5. The laminated assembly of claim 2, wherein said one or more metallic electrodes are obtained by a method comprising hot embossing or stamping a metallic foil onto said polyetherketoneketone film.

6. The laminated assembly of claim 1, wherein an array of electrodes is present on a surface of a polyetherketoneketone film.

7. The laminated assembly of claim 1, additionally comprising a polyetherketoneketone top layer fused to said electrode-bearing layer and encapsulating at least a portion of a plurality of conductive features present on a surface of said electrode-bearing layer.

8. The laminated assembly of claim 1, wherein said polyetherketoneketone is amorphous.

9. The laminated assembly of claim 1, additionally comprising a top layer.

10. The laminated assembly of claim 1, additionally comprising a polyetherketoneketone top layer.

11. The laminated assembly of claim 1, additionally comprising a top layer thermally fused to said electrode-bearing layer.

12. The laminated assembly of claim 1, wherein said laminated assembly is free of any adhesive.

13. The laminated assembly of claim 1, comprising a plurality of conductive features which have been applied to a polyetherketoneketone film by hot stamping or hot embossing.

14. The laminated assembly of claim 1, having a thickness of from about 25 to about 500 microns.

15. The laminated assembly of claim 1, wherein said laminated assembly is flexible.

16. The laminated assembly of claim 1, wherein said electrode-bearing layer has a plurality of electrodes and at least one multiplexer supported thereon wherein said at least one multiplexer is electrically connected to at least two electrodes.

17. The laminated assembly of claim 1 additionally comprising a top layer having at least one opening therein that is positioned to expose at least one electrode.

18. The laminated assembly of claim 1, wherein said electrode-bearing layer has a plurality of electrodes and at least one signal conditioner or amplifier supported thereon wherein said at least one signal conditioner or amplifier is electrically connected to at least two electrodes.

19. The laminated assembly of claim 1, wherein said polyetherketoneketone is semicrystalline.

20. The laminated assembly of claim 1, comprising a plurality of conductive features which have been formed on a polyetherketoneketone film by a process comprising printing.

21. An implantable medical device comprised of the laminated assembly of claim 1.

22. An electrode array useful in an implantable medical device comprising at least first and second implantable and biocompatible polymeric layers, with at least one of said polymeric layers being comprised of polyetherketoneketone, wherein a plurality of patterned conductive features and optionally one or more other electronic components is sandwiched therebetween.

23. A polyetherketoneketone film having a plurality of patterned conductive features, including two or more electrodes, on at least one surface of said polyetherketoneketone film.

24. A method of making an electrode array, said method comprising forming a plurality of patterned conductive features including a plurality of electrodes on a surface of a first polyetherketoneketone film and applying a second polyetherketoneketone film to said first polyetherketoneketone film so as to cover at least some of said patterned conductive features.
